(19) United States
(12) Patent Application Publication
(10) Pub. No.: US 2016/0123980 A1
(43) Pub. Date: May 5, 2016

(54) MULTICOLOR FLOW CYTOMETRY METHOD FOR IDENTIFYING A POPULATION OF CELLS, IN PARTICULAR MESENCHYMAL STEM CELLS

(71) Applicant: CELL THERAPY LIMITED, Singleton Park, Swansea (GB)

(72) Inventors: Martin John Evans, Swansea (GB); Ajan Reginald, Swansea (GB); Sabena Sultan, Swansea (GB)

(21) Appl. No.: 14/892,547

(22) PCT Filed: May 20, 2014
(86) PCT No.: PCT/GB2014/051538

§ 371 Date: Nov. 19, 2015

(30) Foreign Application Priority Data
May 20, 2013 (GB) 1309057.6

(51) Int. Cl. G01N 33/569

(52) U.S. Cl. CPC G01N 33/56966; G01N 2333/705

(57) ABSTRACT

This invention is in the field of the identification and even isolation of mesenchymal stem cells (MSCs) and other cell types by means of differential specific fluorescence activated cell sorting (FACS).

### FIELD OF THE INVENTION

This invention is in the field of the identification and even isolation of mesenchymal stem cells (MSCs) and other cell types by means of differential specific fluorescence activated cell sorting (FACS). That is, the invention is a reagent or composition of matter at various relative specific concentrations, which surprisingly provide for a medicinal (or physiological) effect upon the label cells due to the various specific concentrations of the labeled cell markers in the reagent. The invention is reagents and methods for selective isolation of cells by virtue of the effect of specific relative reagent concentration. The inventors provides a surprising means by which a composition of matter can act distinctively on cells as a drug allowing for their simultaneous identification using fluorescence activated cell sorting (FACS) in the presence or absence of seven or more cell surface markers.

### INTRODUCTION

Mesenchymal stem cells (MSCs) have received much attention for their potential in the field of regenerative medicine and are being used in clinical trials for multiple disorders. MSCs have shown potential for therapeutic benefit in patients with acute myocardial infarction [1, 2], stroke [3], multiple system atrophy (MSA) [4], graft-versus-host disease [5], and spinal cord injury [6]. The International Society for Cellular Therapy (ISCT) developed a set of minimal criteria to be used to identify MSCs [7]. These are: i) adherence to plastic, ii) differentiation into adipocytes, chondrocytes and osteocytes, and iii) a specific expression pattern of cell surface molecules. The two first criteria are evaluated in cell culture under different conditions, whereas the latter can be evaluated using multicolor flow cytometry (MFC). To be identified as MSCs, more than 95% of the cell population must express CD73 (ecto 5′-nucleotidase), CD90 (Thy-1) and CD105 (endoglin), and be negative (≦2% positive cells) for CD11b or CD14, CD34, CD45, CD19 or CD79α, and HLA-DR [7].

The surface marker pattern of MSCs has been analyzed traditionally using a parallel-tube approach: multiple sample tubes with a combination of two to four antibodies each conjugated to a different fluorophore per tube. This involves practical limitations. For instance, the results from parallel tubes are analysed as if the cell population was homogenous and the percentage expression of the surface markers is reported.

The published examples of multicolour panels for MSCs to date either disregard the ISCT-criteria of recommended surface markers (Martins et al., 2009, Jones et al., 2006), use a parallel-tube rather than single-tube approach (Tucker and Bunnell, 2011), or utilise custom conjugates and the most common fluorochromes (Bradford and Clarke, 2011) yielding an inflexible tool that cannot be adapted for individual research requirements. In some cases the researchers have not confirmed that the cells identified as MSCs by flow cytometry also observe the other two minimal criteria: adherence to plastic and tri-lineage differentiation capacity (Martins et al., 2009).

### SUMMARY OF THE INVENTION

The inventors have surprisingly developed a Multicolour Flow Cytometry (MFC) panel including ISCT-recommended mesenchymal stem cell MSC markers on plastic-adherent cells proven to differentiate into adipocytes, chondrocytes and osteocytes. The inventors have designed a core panel that did not use reagents conjugated to the three most commonly available fluorochrome conjugates, fluorochromes FITC (fluorescein isothiocyanate), PE (phycoerythrin) and APC (allophycocyanin), enabling these three positions to be used as interchangeable placeholders and allowing use of the panel in combination with other antigens of interest. The inventors have validated the panel using MSCs from two commonly used tissue sources: umbilical cord matrix and BM aspirate. The inventors have also ensured the panel could be used to identify MSCs within heterogeneous populations.

Multicolour Flow Cytometry (MFC), as opposed to single-colour flow cytometry, introduces a higher technical difficulty in assay development. To analyse several surface markers simultaneously, each surface marker requires a specific antibody for detection. In flow cytometry it is best to use antibodies directly conjugated to fluorochromes instead of primary antibodies for detection and secondary antibodies for signal amplification. Therefore, when using multiple antibodies simultaneously, their conjugated fluorochromes must be chosen wisely so that they do not overlap in their emitted wavelengths. Simply put, the fluorochromes that are as far apart as possible in the colour spectra should be chosen. Success depends mainly on three factors: optimally titrated antibodies, accurate compensation and antigen-fluorochrome balancing. How this is achieved is discussed in more detail below.

The method of the invention has several advantages over the parallel-tube approach. Firstly, increased accuracy for identifying cellular phenotypes at a single cell level is crucial for accurate quantification of specific cell types in patient samples or therapeutic cell products in the clinical setting. It also provides increased sensitivity in detection, enabling MSCs to be identified in mixed cell samples using sophisticated analytical strategies. This is a particular advantage when studying clinical samples that may be heterogeneous. Secondly, single tube flow cytometry enables maximum information to be obtained from small samples such as biopsies or paediatric specimens, and it also facilitates larger research projects since only one tube is required per study subject. Finally a multicolour approach facilitates the detection of different types of MSCs, and allows simultaneous analysis of phenotype and functionality such as cytokine production, apoptosis, and cell cycle analysis (De Rosa et al., 2003).

The invention provides high throughput cellular drug discovery technology which identifies and isolates/captures novel cell types and an algorithm in combination with high throughput cell sorting/automated cell screening. The invention enables the automation and computational analysis of 1000s of markers, colours and marker-colour combinations to identify novel cells types by using the algorithm to identify candidate cells, for example to identify novel cells with the 7 classic MSC markers but also +ve for, for example, CD62. Using the algorithm below, it is possible to define the FACS experiments, test combinations of markers, antibodies and colours to identify/capture the candidate cell if it exists. This process allows a “rational” drug design approach to be applied for the first time to cellular medicines—enabling researchers the design of and empirical testing for novel cell types. Thus enabling researchers in practice to construct (i) a theoretically optimal cell, for example one that expresses the 7 classic MSCs markers and CXCR4 and CD62, (ii) to compute the potential FACS panels to test to isolate this cell (if it exists) and then (iii) to isolate/capture this cell from a 1000s of tissue samples in combination with automated sorting and (iv) critically to test and prove the novelty of the candidate cells through its unique FACS marker profile. Without the invention this process (i-iv) requires months of ‘trial and error’ research involving 100s of laboratory experiments. Using the invention's combination of composition and algorithm enables researchers to automate and rationalize cellular drug discovery and to harness computational approaches (including super) computing for faster, more accurate and more efficient and effective identification and capture of novel cells.

There are several aspects to the present invention. The mesenchymal stem cell (MSC) immunoflorescent labeling reagent comprises a conjugate of a monoclonal antibody to MSCs expressing CD73 (ecto 5′-nucleotidase), CD90 (Thy-1) and CD105 (endoglin), and negative (≦2% positive cells) for CD11b or CD14, CD34, CD45, CD19 or CD79α, and HLA-DR [7] which have been experimentally selected for relative specific concentrations which produce a medicinal effect which presents a specific phenotype which allows for improved identification and even sample collection of specific cell types. One aspect of the invention is as a reagent medicine (drug) to alter the phenotypic expression of the subject cells in a detection assay, that is, as a reagent for both immunoflorescent labeling of mesenchymal stem cells (MSCs) to better characterize said cells by means of selectively varying the respective concentrations of the immunoflorescent antibody labels in the various assay and laboratory reagent comprising antibodies include but not limited to those directed to the following cell types: fibroblast (CD10, CD29, CD106); VSELS (Sca-1, CD45R, Gr-1, TCR alpha-beta, TCR gamma-delta, CD11b, Ter119, Oct-4; HSCs: CD133); EPCs (KDR, VE-cadherin, CD31); MSC Subtypes (CD181, CD184); and tissue-committed stem cells (CD117+, CD184, c-met, AC133). Additionally kits for carrying out these procedures are another aspect.

In the present invention, the novel FACS reagent, is not just used to identify cells which express a particular marker but unexpectedly provides a composition of matter which influences cell phenotype allowing for the ready identification and even isolation of mesenchymal stem cell (MSC) in a cell population by means of altering the respective concentrations of the (active components), that is but is not limited to, the conjugates of the monoclonal antibodies to MSCs. Hereby active components of the reagent, allow for the simultaneous identification over a range of concentrations various MSCs using fluorescence activated cell sorting (FACS). Thereby the reagent(s) allow for the differential detection of MSCs in populations which displays detectable levels of CD73, CD90 and CD105 and do not display detectable levels of CD14, CD19, CD34 and CD45 on its surface and isolating the cell with that surface marker pattern. The invention also provides a reagent for identifying and isolating by virtue of the measured relative concentration of its reactive components, a specific cell type in a population of cells, comprising simultaneously identifying using FACS the presence or absence of seven or more cell surface markers indicative of the specific cell type on the surfaces of the cells in the population and isolating the cell with the indicative cell surface markers. Wherein said reagent can be used in vivo, in vitro and ex vivo to act in humans or animals and to act on stem and non-stem cells including but not limited to being used in combination with media, excipients, inactive additives, blood, tissues and other bodily fluids; including but not limited to use in combination with flow cytometry whereas its uses can include but not be limited to the isolation, detection and to aid in the collection of cells from tissue, blood, solutions and solids including frozen samples, and pathology specimens.

The inventors herein provide a surprising means by which a composition of matter can act on cells as a drug allowing for users to simultaneously identify using fluorescence activated cell sorting (FACS) the presence or absence of seven or more cell surface markers.

An algorithm and panel to phenotype, that is but is not limited to a mean to identify, isolate, and distinguish Mesenchymal Stem Cells (MSCs) from other cells in peripheral blood, bone marrow, adipose, biopsy sample, or isolated from any other tissues or engineered or resulting from IPS by means of transdifferentiation is hereby proposed. We provide for the improved prediction of the phenotype of MSC by a fluorophore ISCT core panel and algorithmic means by which to better predict MSC phenotype. MSCs possess the potential for use in the field of regenerative medicine therapies and are being used in clinical trials for multiple disorders. Thereby the International Society for Cellular Therapy (ISCT) has developed a set of minimal criteria to be used to identify MSCs, including a specific pattern of cell surface molecule expression measured by multicolor flow cytometry (MFC). The MFC methodology for surface marker expression of MSCs has traditionally used multiple parallel samples. There are many advantages to being able to perform a single-sample multicolor analysis of MSCs. In this study we report development of a 10-fluorophore panel consisting of a 7-fluorophore ISCT core panel for identification of MSCs, and 3 placeholder positions which can be used for reagents to identify different subtypes of MSCs. This panel can be used to phenotype bone marrow and umbilical cord matrix MSCs, and distinguish rare events of MSCs in mixed populations of fibroblasts and peripheral blood mononuclear cells. This tool offers a valuable method to identify and quantify MSCs in blood and bone marrow, and could also be used for single-cell suspensions from digested tissues.

The inventors have surprisingly shown that it is possible to simultaneously identify using fluorescence activated cell sorting (FACS) the presence or absence of seven or more cell surface markers.

The invention provides a method of identifying, and optionally isolating, a MSC in a cell population, comprising simultaneously identifying using FACS a cell in the population which displays detectable levels of CD73, CD90 and CD105 and does not display detectable levels of CD14, CD19, CD34 and CD45 on its surface and thereby identifying, and optionally isolating, a mesenchymal stem cell (MSC) in a cell population. The presence or absence of the seven markers is identified simultaneously using FACS. The presence or absence of the seven markers is identified at the same time. The method of the invention uses one sample of the population. It does not involve a parallel-tube approach.

The invention also provides a method of identifying, and optionally isolating, a specific cell type in a population of cells, comprising simultaneously identifying using FACS the presence or absence of seven or more cell surface markers indicative of the specific cell type on the surfaces of the cells in the population.

The invention provides a kit for identifying, and optionally isolating, a mesenchymal stem cell (MSC) in a cell population, comprising seven or more fluorescently-labelled antibodies, wherein at least one antibody in the kit specifically binds to each of CD73, CD90, CD105, CD14, CD19, CD34 and CD45.

The invention also provides a kit for identifying, and optionally isolating, a specific cell type in a population of cells, comprising seven or more fluorescently-labelled antibodies, wherein at least one antibody in the kit specifically binds to on of seven or more cell surface markers indicative of the specific cell type.

The invention provides a reagent and kit for identifying, and optionally isolating, a MSC in a cell population, comprising simultaneously identifying using FACS a cell in the population which displays detectable levels of CD73, CD90 and CD105 and does not display detectable levels of CD14, CD19, CD34 and CD45 on its surface and thereby identifying a mesenchymal stem cell (MSC) in a cell population. The presence or absence of the seven markers is identified simultaneously using FACS.

The invention also provides a reagent for identifying, and optionally isolating, a specific cell type in a population of cells, comprising simultaneously identifying using FACS the presence or absence of seven or more cell surface markers indicative of the specific cell type on the surfaces of the cells in the population.

The invention also provides a method of producing a panel of fluorescently-labelled antibodies for simultaneously identifying the presence or absence of seven or more cell surface markers using FACS analysis, the method comprising (a) selecting the seven or more cell surface markers; (b) spreading the positive markers on different lasers; (c) selecting seven or more fluorescently-labelled antibodies; (d) titrating the antibodies to ensure that optimal concentration and minimal spectral overlap is achieved on cells with known expression of the seven or more markers (both positive and negative); (e) testing the antibodies against the positive cell surface markers; (f) testing a core panel of the antibodies; (g) inducing or stimulating cells to express the placeholder markers; (h) testing and titrating the placeholder antibodies; (i) optimising the placeholder conditions; and (j) testing the full panel on cells in the absence of other cell types and in mixed populations.

The invention also provides a panel of fluorescently-labelled antibodies for simultaneously identifying the presence or absence of seven or more cell surface markers using FACS analysis produced using the method of the invention.

The invention also provides a method of identifying, and optionally isolating, a novel cell type in a population of cells, comprising simultaneously identifying using FACS the presence or absence of seven or more cell surface markers on the surfaces of the cells in the population, wherein at least some of the seven or more markers are indicative of a specific cell type and at least one of the seven or more markers is not detectably expressed by the specific cell type.

### DESCRIPTION OF THE FIGURES

FIG. 1 shows UCM MSCs stained with the 10-colour panel. A) CD14-FMO used to set the gate so that there are <1% CD14+ events. B) Gate in A modified using the CD19-FMO used to set the CD19+ threshold so that there are <1% CD19+ events. C) Gates applied to the sample stained with the full panel for phenotype analysis.

FIG. 2 shows 6-step gating strategy, shown above in a schematic version as well as exemplified with a sample of UCM MSCs. 1) All un-gated recorded events from the tube stained with all 10 antibodies on a FSC vs. SSC plot. A gate was drawn to exclude debris and doublets. 2) CD14 vs. CD19 plot gating on the CD14−/CD19− events. 3) CD34 vs. CD45 plot gating on the CD34−/CD45− events. 4) CD73 vs. CD90 plot gating on the CD73+/CD90+ events. 5) CD105 vs. SSC plot displaying CD105-intensity. 6) An overlay of unstained and stained cells displaying a clear shift in CD105-intensity, confirming the last positive ISCT-marker. Black=unstained, Red=stained.

FIG. 3 shows core surface marker expression in the presence of the bright or dim placeholders. UCM (n=6) and BM MSCs (n=6) were gated using the gating strategy described earlier. UCM MSCs display a more homogenous forward and side scatter profile than BM MSCs. BM MSCs isolated from BM aspirates obtained from trauma patients display a small CD34dim population (samples #2, #3, #5, #6) compared to commercial BM aspirate (#4) and BM MSCs (#1) and UCM MSCs. All samples express CD 105, when compared to the unstained control (overlay histograms).

FIG. 4 shows differences in placeholder panel expression between BM and UCM MSCs. Samples were stained with either the bright (UCM MSC, n=3; BM MSC, n=3) or the dim 10-colour panel (UCM MSC, n=6; BM MSC, n=4). The placeholder antibodies used in the bright panel were CD29-FITC, CD164-PE and CD44-APC, and the ones in the dim panel were CD49d-FITC, CD29-PE and CD182-APC. Amongst the markers that make up the dim placeholder panel (CD29-PE, CD49d-FITC, CD182-APC) there is only a difference in expression between BM and UCM MSCs in terms of CD49d which is higher expressed in UCM MSCs (p=0.006).

FIG. 5 shows identification of MSCs in a dominant fibroblast mix. UCM-MSCs (2%) were mixed with HSFs. A1) All un-gated recorded events from the tube of UCM MSCs stained with the 10-colour panel. A cell gate was drawn to exclude debris and doublets. B1) All un-gated recorded events from the tube of HSFs. The cell gate included the HSFs. C1) HSFs with 2% MSCs added. At 60,000 recorded cell events MSCs cannot be distinguished from HSFs based on forward and side scatter. C2-C6) MSC gating strategy as shown in FIG. 2, resulting in 2.0% identified MSCs (1,164 MSCs out of 59,550 cell events).

FIG. 6 shows identification of MSCs in a dominant PBMC mix. UCM-MSCs (1%) were mixed with PBMCs. A1) All un-gated recorded events from the tube of UCM MSCs stained with the 10-colour panel. A cell gate was drawn to exclude debris and doublets. B1) All un-gated recorded events from the tube of PBMCs. The cell gate included the PBMCs. C1) PBMCs with 1% MSCs added. At 100,000 recorded cell events MSCs cannot be distinguished from PBMCs based on forward and side scatter. C2-C6) MSC gating strategy as shown in FIG. 2, resulting in 0.63% identified MSCs.

### DETAILED DESCRIPTION

### Cell Type Detection

The method of the invention can be used to identify, and optionally isolate, a variety of specific cell types using their cells surface marker pattern. These include, but are not limited to, mesenchymal stem cells (MSCs), progenitor cells of mesodermal lineage, fibroblasts, very small embryonic/epiblast-like stem cells (VSELs), hematopoietic stem cells (HSCs), endothelial progenitor cells (EPCs) and tissue-committed stem cells. The cell surface marker patterns for these cells are set out in the claims.

The method concerns simultaneously identify using FACS the presence or absence of seven or more cell surface markers. Any number of markers can be simultaneously identified. For instance, the invention may concern identifying using FACS the presence or absence of 8 or more, 9 or more, 10 or more, 12 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or cells, 150 or more, 200 or more, 250 or more, 300 or more, 350 or more, 400 or more, 500 or more, 600 or more, 700 or more, 800 or more, 900 or more, 1000 or more, 1500 or more, 2000 or more, 2500 or more or 5000 or more surface markers. This can be done as discussed in more detail below.

The presence or absence of seven or more cell surface markers is determined simultaneously, i.e. at the same time. Using the invention, FACS analysis may be carried out once on a population of cells and it is possible to determine from this whether or not the seven or more markers are present or absent. It is not necessary to run the FACS analysis more than once or with multiple samples. The invention avoids the use of the parallel-tube approach.

Antibodies that are capable of specifically binding to the various cells surface markers are known in the art. An antibody “specifically binds” to a cell surface marker sequence when it binds with preferential or high affinity to that marker but does not substantially bind, does not bind or binds with only low affinity to other cell surface markers or other proteins. For instance, an antibody “specifically binds” to CD73 when it binds with preferential or high affinity to CD73 but does not substantially bind, does not bind or binds with only low affinity to other cell surface markers or proteins, such as CD90, CD105, CD14, CD19, CD34 and CD45.

An antibody binds with preferential or high affinity if it binds with a Kd of 1×10⁻⁷ M or less, more preferably 5×10⁻⁸ M or less, more preferably 1×10⁻⁸ M or less or more preferably 5×10⁻⁹ M or less. A portion binds with low affinity if it binds with a Kd of 1×10⁻⁶ M or more, more preferably 1×10⁻⁵ M or more, more preferably 1×10⁻⁴ M or more, more preferably 1×10⁻³ M or more, even more preferably 1×10⁻² M or more. A variety of protocols for competitive binding or immunoradiometric assays to determine the specific binding capability of compounds, such as antibodies or antibody constructs and oligonucleotides are well known in the art (see for example Maddox et al, J. Exp. Med. 158, 1211-1226, 1993).

The antibody may be, for example, a monoclonal antibody, a polyclonal antibody, a single chain antibody, a chimeric antibody, a CDR-grafted antibody or a humanized antibody. The antibody may be an intact immunoglobulin molecule or a fragment thereof such as a Fab, F(ab′)₂ or Fv fragment.

Fluorescent labels suitable for use in the method of the invention are discussed below with reference to the kits of the invention.

MSCs display detectable levels of CD73, CD90 and CD105 and do not display detectable levels of CD14, CD19, CD34 and CD45 on their surfaces. This pattern of expression can be detected using the method of the invention. Hence, MSCs can be identified and optionally isolated.

A progenitor cell of mesodermal lineage expresses detectable levels of CD29, CD44, CD73, CD90, CD105 and CD271 and (b) does not express detectable levels of CD14, CD34 and CD45. These cells preferably also express CXCR1, CXCR2 or CXCR4. They more preferably express all of CXCR1, CXCR2 and CXCR4. They are disclosed in International Application No. PCT/GB2012/051600 (published as WO 2013/005053). This pattern of expression can be detected using the method of the invention. Hence, PMLs can be identified and optionally isolated.

The specific cell type is preferably a stromal cells. Other specific stromal cells types that can be identified using the method of the invention and their and their surface marker expression are shown below. + means the cells display detectable levels of the marker. − means the cells do not display detectable levels of the markers. Markers that are shared by mouse and human are indicated (m/h) after the marker, as are those that are unique to mouse (m) or human (h). This is taken from Johanna A. Joyce & Jeffrey W. Pollard, Nature Reviews Cancer 9, 239-252 (April 2009).

Tumor associated macrophage (TAM): CD11b+ CD14+ CD31− CD34− CD45+ CD68+CD117− CD133− CD146− CD204+ CD206+ CCR2+ CSF1R+ MHC II+ VEGFR1+ VEGFR2− (m/h) F4/80+ (m) CD23+ CD163+ CXCR4+.

Myeloid-derived Suppressor Cells (MDSC): CD11b+ CD14+/− MHC I+ MHC II low (m/h) GR1+ CD11b+ and can be further subdivided into LY6G+LY6Clow CD11b+CD11c+/− CD33+CD34+CD86−

Monocytes that express the angiopoietin receptor TIE2 (TEM): CD11b+ CD14+ CD31 low CD34− CD45+ CD117− CD133− TIE2+ VEGFR2− (m/h) F4/80+ GR1 low SCA1− (m) CD11c+ CD13+ CD16+ CD33+ CD62L− CD146− CCR2− CCR5+ CSF1R+ (h)

Neutrophil: CD11b+ CD14 low CD31+ CD66B+ CXCR2+ (m/h) GR1+ VEGFR1+ CXCR1− (m) CD15+ CXCR1+ (h)

Mast cell: CD43+ CD117+ CD123+ CD153+ (m/h) CD11b+ CD16+ CD34+ SCA1+ (m) CCR1+ CCR3+ CCR4+ CCR5+ CXCR1+ CXCR2+ CXCR4+ (h)

Endothelial cell: CD31+ CD34+ CD105+ CD106+ CD144+ (m/h)

Pericyte: Desmin+/− NG2+/− SMA+/−PDGFR+/−

Fibroblast: Vimentin+ desmin+SMA+/−FSP1+FAP+(m/h)

Platelet: CD41+ CD42a-d CD51+ CD110+ (m/h)

CD4+ T cell CD3+ CD4+ CD45+ (m/h)

CD8+T cells CD3+ CD8+ CD45+ (m/h)

B cell: CD3− CD19+ CD20+ CD45+ (m/h) CD45RA+ B220+ (m)

NK cell: CD11b+ CD27+ CD3− CD16+/− CD56+ CD3− CD335+ NKp46+ (m/h)

The invention also provides a method of identifying, and optionally isolating, a novel cell type. The method comprises simultaneously identifying using FACS the presence or absence of seven or more cell surface markers on the surfaces of the cells in the population, wherein at least some of the seven or more markers are indicative of the specific cell type and at least one of the seven or more markers is not detectably expressed by the specific cell type. The specific cell type may be any of those discussed above. The specific cell type is preferably a MSC. The method may concern simultaneously identifying using FACS the presence or absence of eight or more cell surface markers on the surfaces of the cells in the population, wherein the eight or more markers comprise CD73, CD90, CD105, CD14, CD19, CD34 and CD45 and at least one of the eight or more markers is not expressed by MSCs. The cell surface markers on MSCs have been well characterized and it is routine to identify at least one cell surface marker that is not expressed by MSCs. A skilled person can design a panel of markers and corresponding antibodies using the algorithm below to identify novel cell types in a high throughput manner.

FACS analysis is a well known technique. One way of performing this technique is disclosed in the Examples. FACS can isolate single cells. The method of the invention may be for identifying, and optionally isolating, a single cell of a particular type, such as a single MSC or a single progenitor cell of mesodermal lineage, or a novel cell type as discussed above. Alternatively, the method of the invention may be for identifying, and optionally isolating, two or more cells of a particular type, such as two or more MSCs or two or more progenitor cells of mesodermal lineage.

FACS may also isolate cells which express particular markers. Hence, the invention also provides a method of identifying and isolating a mesenchymal stem cell (MSC) in a cell population, comprising simultaneously identifying using fluorescence activated cell sorting (FACS) a cell in the population which displays detectable levels of CD73, CD90 and CD105 and does not display detectable levels of CD14, CD19, CD34 and CD45 on its surface and isolating the cell with that surface marker pattern. The invention also provides a method of identifying and isolating a specific cell type in a population of cells, comprising simultaneously identifying using FACS the presence or absence of seven or more cell surface markers indicative of the specific cell type on the surfaces of the cells in the population and isolating the cell with the indicative cell surface markers.

The antibodies used in the FACS method are typically titrated for use in the method as discussed below.

The cell population is typically present in a sample. The sample is preferably a fluid sample. The sample typically comprises a body fluid of the patient. The sample may be urine, lymph, saliva, mucus, milk or amniotic fluid but is preferably blood, plasma or serum. The sample may be from bone marrow. Typically, the sample is human in origin, but alternatively it may be from another mammal animal such as from commercially farmed animals such as horses, cattle, sheep or pigs or may alternatively be pets such as cats or dogs.

The sample is typically processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

### Kit and Reagant

The invention also provides kits for identifying, and optionally isolating, a mesenchymal stem cell (MSC) or a specific cell type. The kit of the invention may be for identifying, and optionally isolating, a single cell of a particular type, such as a single MSC or a single progenitor cell of mesodermal lineage, or a novel cell type. Alternatively, the kit of the invention may be for identifying, and optionally isolating, two or more cells of a particular type, such as two or more MSCs or two or more progenitor cells of mesodermal lineage or a MSC and a progenitor cell of mesodermal lineage.

The kit comprises seven or more fluorescently-labelled antibodies. At least one antibody in the kit specifically binds each of the cell surface markers being used to in identify, and optionally isolate, the MSC or specific cell types. For instance, in one embodiment, the kit comprises seven or more fluorescently-labelled antibodies and at least one antibody in the kit specifically binds to each of CD73, CD90, CD105, CD14, CD19, CD34 and CD45. Other specific kits are described in the claims.

Specific binding is discussed above.

The kits of the invention may be used in the methods of the invention to identify, and optionally isolate, a MSC or specific cell type in a population of cells using FACS. The kit may be for identifying, and optionally isolating, any of the cells types discussed above.

Antibodies against the various markers are commercially available. Table 1 summarises commercial sources for antibodies against CD11b, CD14, CD19 and CD79α.

Each antibody in the kit is typically labeled with a different fluorescent label. The seven or more fluorescent labels are typically chosen such that they can be identified using FACS. The seven or more fluorescent labels are preferably selected from BV605, K-Orange, eF450, PE-Cy7, PerCP-Cy5.5, PE, FITC/AF488, APC-eF780, AF700 and APC. The seven or more fluorescent labels are preferably selected from BV605, K-Orange, eF450, PE-Cy7, PerCP-Cy5.5, APC-eF780 and AF700. Any combination of these labels may be used. Suitable FACS configuration for use with these labels are shown below.

The seven or more fluorescently-labelled antibodies preferably comprise anti-CD14-APC-eFluor780 (clone 61D3, eBioscience, Hatfield, Ireland, UK), anti-CD19-PE-Cy7 (clone J3-119, Beckman Coulter, London, UK), anti-CD34-PerCP-Cy5.5 (clone 581, BioLegend, San Diego, Calif., US), anti-CD45-Krome Orange (clone J.33, Beckman Coulter), anti-CD73-eFluor450 (clone AD2, eBioscience), anti-CD90-AlexaFluor700 (clone 5E10, BioLegend), anti-CD105-Brilliant Violet 605 (clone 266, BD Bioscience, Oxford, UK). All antibodies were mouse isotype IgG1, κ.

Each of the seven or more fluorescently-labelled antibodies are typically titrated to an appropriate concentration for use in the FACS method of the invention. Each antibody is typically titrated at 1:10 to 1:1000000, such as 1:50, 1:100, 1:500, 1:1000, 1:10,000, 1:50,000, 1:100,000. Titration is important in the method of the invention as discussed in detail below.

The kits of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), and/or other reagents needed for FACS analysis. Reagents may be present in the kit in a dry state such that a fluid sample resuspends the reagents. The kit may also, optionally, comprise instructions to enable the kit to be used in the method of the invention or details regarding which patients the method may be used for.

### Algorithm

The invention allows the presence or absence of seven or more cell surface markers to be identified simultaneously. In order to do this, several factors have to be taken into account using the following algorithm.

The purpose of the invention is to (i) develop a multicolour flow cytometry panel including ISCT-recommended positive and negative surface markers on plastic-adherent cells; (ii) design a panel that does not use reagents conjugated to the three most common fluorochromes FITC (fluorescein isothiocyanate), PE (phycoerythrin) or APC (allophycocyanin) enabling these three positions to be used as interchangeable placeholders allowing researchers to use the panel in combination with other antigens of interest; and (iii) validate the panel using cells, such MSCs from umbilical cord matrix and bone marrow aspirate.

The success of the method depends on (i) the number of markers selected to be identified; (ii) the number of fluorescence channels/detectors in the FACS machine; (iii) the number of lasers on each channel/detector of the FACS machine; (iv) the number of antibodies available for each marker; (v) the sensitivity of an antibody (how bright the signal is in the region of the spectrum); (vi) the performance of the antibody conjugate (how bright the fluorochrome is and the expression level of the marker); and (vii) the presence of the other antibodies on the same cell. These all need to be taken into account when designing a suitable panel of fluorescently-labelled antibodies.

The invention provides a method of producing a panel of fluorescently-labelled antibodies for simultaneously identifying the presence or absence of seven or more cell surface markers using FACS analysis, the method comprising (a) selecting the seven or more cell surface markers; (b) spreading the positive markers on different lasers; (c) selecting seven or more fluorescently-labelled antibodies; (d) titrating the antibodies to ensure that optimal concentration and minimal spectral overlap is achieved on cells with known expression of the seven or more markers (both positive and negative); (e) testing the antibodies against the positive cell surface markers; (f) testing a core panel of the antibodies; (g) inducing or stimulating cells to express the placeholder markers; (h) testing and titrating the placeholder antibodies; (i) optimising the placeholder conditions; and (j) testing the full panel on cells in the absence of other cell types and in mixed populations.

Step (b) ensures minimal spectral overlap and interference with other markers. The different lasers are in the FACS machine. The seven or more fluorescently-labelled antibodies in (c) specifically bind to the seven or more markers. Typically, there is one antibody which specifically binds to each of the seven or more markers. However, the panel may comprise more than one antibody which specifically binds to one or more of the seven or more markers. Suitable antibodies and fluorescent labels are discussed above.

Titration in step (d) can be carried out using routine methods. Suitable concentration of antibodies for use in the invention are discussed above. The concentrations of the seven or more antibodies are tested using FACS analysis to ensure minimal spectral overlap and interference with the other antibodies which specifically bind to the other markers.

Steps (e), (f), (h), (i) and (j) are typically carried out using FACS analysis. This allows antibodies to be tested and titrated and conditions to be optimised.

The core panel of antibodies in (f) specifically bind to a core panel of cell surface markers for the specific cell type of interest, such as any of the core panels discussed above. For MSCs, the core panel of seven antibodies, which specifically bind to CD73, CD90, CD105, CD14, CD19, CD34 and CD45, may be tested on cell lines of MSCs that are commercially available and mixed populations thereof (i.e. non MSCs and MSCs). The 10 colour panel discussed above and in the Examples may also be tested under both of these conditions. This is what was done in the Examples. The core panel of fluorescently-labelled antibodies typically does not comprise FITC (fluorescein isothiocyanate), PE (phycoerythrin) and APC (allophycocyanin).

The placeholder antibodies in (g) may comprise FITC (fluorescein isothiocyanate), PE (phycoerythrin) and APC (allophycocyanin). The interchangeable placeholder antibodies allow the core panel to be tested in combination with other cell surface markers of interest. For instance, for MSCs, the core panel of seven antibodies, which specifically bind to CD73, CD90, CD105, CD14, CD19, CD34 and CD45, may be tested in combination with other cell surface markers, such as CD62. This allows new cell types to identified as discussed above.

The reason for titrating antibodies for flow cytometry as in step (d) and/or (h) is to allow optimal separation between positive and negative signals without unnecessarily wasting antibody, thus reducing background noise and the overall cost of the method. Too high antibody concentrations in the staining volume can also lead to non-specific antibody binding. Titration is therefore good practice, not only to increase specificity of the assay but also to reduce reagent consumption and thus cost (ICSH/ICCS, 2013). The goal of the titration is to identify the antibody concentration that results in the highest stain index. This is routine in the art.

The method of producing a panel of fluorescently-labelled antibodies in accordance with the invention can be automated. An automated method removes manual/human processing constraints and allows automated screening and computational power to enable rationale high throughput cellular drug discovery. This allows a panel of fluorescently-labelled antibodies for simultaneously identifying the presence or absence of 1000 or more cell surface markers using FACS analysis to be produced.

The invention also provides a panel of fluorescently-labelled antibodies for simultaneously identifying the presence or absence of seven or more cell surface markers using FACS analysis produced using the method of the invention.

The invention also provides a method of identifying a mesenchymal stem cell (MSC) in a cell population, comprising producing a panel of fluorescently-labelled antibodies for simultaneously identifying using fluorescence activated cell sorting (FACS) the presence of CD73, CD90 and CD105 and the absence of CD14, CD19, CD34 and CD45 using (b) to (j) above and identifying using fluorescence activated cell sorting (FACS) a cell in the population which displays detectable levels of CD73, CD90 and CD105 and does not display detectable levels of CD14, CD19, CD34 and CD45 on its surface using the panel of fluorescently-labelled antibodies and thereby identifying a mesenchymal stem cell (MSC) in a cell population.

The invention also provides a method of identifying a specific cell type in a population of cells, comprising producing a panel of fluorescently-labelled antibodies for simultaneously identifying using fluorescence activated cell sorting (FACS) the presence of seven or more cell surface markers in the specific cell type using (a) to (j) above and simultaneously identifying using FACS the presence or absence of the seven or more cell surface markers indicative of the specific cell type on the surfaces of the cells in the population using the panel of fluorescently-labelled antibodies.

The method may comprise producing a panel of fluorescently-labelled antibodies for identifying the presence or absence of ten or more cell surface markers indicative of MSCs or the specific cell type. Any of the embodiments discussed above equally apply to this embodiment.

One particular embodiment of the present invention is directed toward an algorithm, and method to: (i) develop an MFC panel including ISCT-recommended MSC markers on plastic-adherent cells proven to differentiate into adipocytes, chondrocytes and osteocytes; (ii) design a core panel that did not use reagents conjugated to the three most commonly available fluorochrome conjugates—FITC (fluorescein isothiocyanate), PE (phycoerythrin) or APC (allophycocyanin) enabling these three positions to be used as interchangeable placeholders allowing researchers to use the panel in combination with other antigens of interest; (iii) validate the panel using MSCs from two commonly used tissue sources: umbilical cord matrix and bone marrow aspirate; (iv) ensure the panel could be used to identify MSCs within heterogeneous populations.

Another particular embodiment is directed toward the development of a better means to process the data from an MFC panel includes the items of sample data using an aggregated classification and regression tree model formed using a statistical ensemble or committee method such as a bootstrap, bagging, or arcing algorithm. In a further embodiment, the aggregated classification and regression tree model is trained by preprocessing.

i) historical data comprising actual sample data of samples previously recorded for percent MSC content, and

ii) the corresponding favorable post-MSC analysis outcomes for those samples to learn how to predict favorable post-MSC analysis outcomes. In one particular embodiment, the preprocessing comprises reducing the quantity of the historical sample data and corresponding sample favorable post-MSC analysis outcomes; reducing the number of variables contained in the historical sample data and corresponding sample favorable post-MSC analysis outcomes using classification and regression trees; transforming the values of the historical sample data and corresponding sample favorable post-MSC analysis outcomes; applying a boosting algorithm to the extracted features; and generating the classification and regression tree model to predict a favorable outcome (high MSC yield) from the boosted extracted features. In one further embodiment, the model is cross-validated by repeated training of the model in a randomly chosen 90 percent training sample followed by prediction in the remaining 10 percent hold-out test set to yield estimates of the screening-related improvement in favorable post-MSC analysis outcomes.

In still another embodiment, the prediction of the favorable outcome (good yield) comprises processing the items of data using an aggregated classification and regression tree model formed using a bootstrap algorithm including a combination of many distinct trees, each model estimated in a sequence of bootstrap samples drawn from the original sample, and wherein a screening decision used to calculated the phenotype of MSC cells to predicted for a sample is then based on a combination of average predicted favorable across bootstrap trees and a majority vote criterion comprising whether a majority of the bootstrap trees predict favorable above a predetermined threshold level.

In one particular embodiment, the development of a better means to process the items of sample information using an aggregated classification and regression tree model formed using a statistical ensemble or committee method such as a bootstrap, bagging, or arcing algorithm. In a further embodiment, the aggregated classification and regression tree model is trained by preprocessing.

i) historical data comprising actual sample information of samples previously recorded for percent MSC content, and
ii) the corresponding favorable post-MSC analysis outcomes for those samples to learn how to predict favorable post-MSC analysis outcomes. In one particular embodiment, the preprocessing comprises reducing the quantity of the historical sample data and corresponding sample favorable post-MSC analysis outcomes; reducing the number of variables contained in the historical sample data and corresponding sample favorable post-MSC analysis outcomes using classification and regression trees; transforming the values of the historical sample data and corresponding sample favorable post-MSC analysis outcomes; applying a boosting algorithm to the extracted features; and generating the classification and regression tree model to predict a favorable outcome (high MSC yield) from the boosted extracted features. In one further embodiment, the model is cross-validated by repeated training of the model in a randomly chosen 90 percent training sample followed by prediction in the remaining 10 percent hold-out test set to yield estimates of the screening-related improvement in favorable post-MSC analysis outcomes.

In another embodiment, the prediction of the favorable outcome comprises processing the items of data using an aggregated classification and regression tree model formed using a bootstrap algorithm including a combination of many distinct trees, each model estimated in a sequence of bootstrap samples drawn from the original sample, and wherein a screening decision used to calculated the number of MSC cells to predicted for a sample is then based on a combination of average predicted favorable across bootstrap trees and a majority vote criterion comprising whether a majority of the bootstrap trees predict favorable above a predetermined threshold level.

### EXAMPLE

### Materials and Methods

### Collection of Human Umbilical Cord Matrix Samples

Human umbilical cords and placentas were collected from full-term births after elective caesarean section delivery and aseptically stored at room temperature during transport for less than 90 min from delivery until processing. The umbilical cord was separated from the placenta and a 10 cm section proximal to the placenta was removed and placed in a sterile container. The cord was rinsed with phosphate buffered saline (PBS; Life Technologies Ltd, Paisley, UK) to wash away the blood, and incubated in Hanks buffered salt solution (HBSS) supplemented with Antibiotic-Antimycotic (both Life Technologies) for 2 h at 4° C. This study was approved by the local research ethics committee and all mothers gave informed written consent. Umbilical cord matrix MSCs were prepared according to published methods [13, 14] with some modifications. Cells were then harvested, counted, and cryopreserved in passage 3 in culture media supplemented with 10% dimethyl sulfoxide (Sigma Aldrich, Poole, UK) to −80° C. and stored in liquid nitrogen for later use.

### Collection of Human Bone Marrow Aspirate Samples

Bone marrow aspirate was collected undergoing surgery for orthopedic trauma. A 1-8 ml sample was collected in heparinized syringes from the iliac crest and stored at room temperature during transport for less than 1 h until processing. The bone marrow was diluted 1:2 with HBSS (Life Technologies) and layered over Ficoll-Paque PLUS 1.077 (GE Healthcare, Uppsala, Sweden) for isolation of bone marrow mononuclear cells (BMMCs) by centrifugation. This study was approved by the local research ethics committee and the patients gave informed written consent. BMMCs were seeded at 1×105 cells/cm2 in T25 flasks (CellSTAR, Greiner Bio-One, Stonehouse, UK) in 5 ml of cell culture media, αMEM (Life Technologies), 10% platelet lysate prepared from expired apheresis platelets, 2 mM GlutaMAX (Life Technologies), 1% Penicillin-Streptomycin (Life Technologies), 5 U/ml Heparin (CalBiochem, Merck KGaA, Darmstadt, Germany), and incubated at 37° C., 5% CO2-in-air. On day 8 the cells were harvested after removal of spent media by covering the culture surface with Accutase (Sigma Aldrich) and incubating the flask for 5 min at 37° C. to detach the cells. Cells were collected by centrifugation, re-suspended in complete media, and counted as above; cells were then cultured at 500 cells/cm2 until 95% confluent. Cells were harvested, counted, and cryopreserved in passage 2 in culture media/10% DMSO as above. One additional bone marrow sample from a young healthy volunteer (22-year-old male) as well as isolated MSCs cryopreserved in passage 2 (22-year-old female) were purchased from Lonza (Lonza, Slough, UK) as an internal control of the processing and cell isolation protocol.

### Human Fibroblast Cell Line

The human skin fibroblast (HSF) cell line 1184 was used as a negative control to test whether or not it was possible to use the panel to identify MSCs in a mixed cell population. The cells were cultured in high-glucose DMEM (Sigma Aldrich), 10% FCS (Sigma Aldrich), 2 mM GlutaMAX (Life Technologies), 1% Penicillin/Streptomycin (Life Technologies) until 80-90% confluent. They were harvested using Accutase as above.

### Peripheral Blood Mononuclear Cells (PBMCs)

Peripheral blood from healthy volunteers was collected in heparinized vacutainer tubes (Greiner Bio-One) after informed written consent with approval from the local research ethics committee. The blood sample was diluted 1:2 with HBSS (Invitrogen), layered on Ficoll-Paque PLUS 1.073 (GE Healthcare) and the PBMCs were isolated by centrifugation. The cells were counted as above and mixed with MSCs to evaluate if the panel could identify MSCs in a heterogeneous sample.

### MSC Preparation

UCM and BM MSCs were thawed and propagated in their respective culture media described above, at 5,000 cells/cm2, in 5% CO2-in-air at 37° C., and harvested using Accutase. UCM MSCs and BM MSCs were used in passage 5. The cellular viability at harvest was never less than 82%.

### Surface Marker Selection

### Positive Markers

According to the ISCT-criteria MSCs must express CD73, CD90 and CD105 so these were included in the multicolor panel.

### Negative Markers

Of ISCT's negative markers, the following were included: CD14, CD19, CD34, and CD45. Because HLA-DR expression by MSCs can be induced, it was not considered a definitive negative marker and was thus omitted from the panel [7]. Of the two monocyte markers recommended by the ISCT, CD11b and CD14, CD14 was chosen because of the greater variety of products available that target this antigen, especially the variety of fluorophores to which anti-CD14 antibodies are conjugated. The B lymphocyte marker CD19 was chosen over CD79α for the same reason. Thirteen suppliers were reviewed in July 2012—CD11b: 79 products, CD14: 100 products, CD19: 108 products, CD79α: 26 products, see Table 1.

### Monoclonal Antibody Selection

### 7-Colour Core Panel

The criteria for identifying monoclonal antibodies targeting the selected surface markers above was that they should not be conjugated to FITC, PE and APC so that these commonly used fluorochromes would be available to study additional markers of interest. Similarly, it was desired that the violet laser be used to its full potential (3×photomultipliers for BD FACSAria I available to us) as fluorophores suitable for the violet laser are less commonly available. Furthermore, it was decided to spread the positive markers on different lasers to minimise spectral overlap and interference with the other markers. With these criteria in mind, 13 suppliers were screened for their fluorophore-conjugated monoclonal antibodies against CD14, CD19, CD34, CD45, CD73, CD90 and CD105. The monoclonal antibodies selected for use in development of the panel were: anti-CD14-APC-eFluor780 (clone 61D3, eBioscience, Hatfield, Ireland, UK), anti-CD19-PE-Cy7 (clone J3-119, Beckman Coulter, London, UK), anti-CD34-PerCP-Cy5.5 (clone 581, BioLegend, San Diego, Calif., US), anti-CD45-Krome Orange (clone J.33, Beckman Coulter), anti-CD73-eFluor450 (clone AD2, eBioscience), anti-CD90-AlexaFluor700 (clone 5E10, BioLegend), anti-CD105-Brilliant Violet 605 (clone 266, BD Bioscience, Oxford, UK). All antibodies were mouse isotype IgG1, κ.

### Placeholders

To test the placeholder concept the following reported positive MSC-markers were used in combination with the core panel: anti-CD29-FITC (clone TS2/16, eBioscience), anti-CD29-PE (clone HUTS-21, BD Bioscience) [15], anti-CD44-APC (clone IM7, eBioscience) [16], anti-CD49d-FITC (clone 44H6, Serotec, Kidlington, UK) [17], anti-CD164-PE (clone N6B6, BD Bioscience) [18], and anti-CD182-APC (clone 5E8/CXCR2, BioLegend) [19]. All antibodies were mouse isotype IgG1, κ, apart from CD44-APC (IgG2b, κ), CD29-PE and CD164-PE (both IgG2α, κ). The placeholders were divided into two panels based on signal intensity from preliminary data. One bright panel, CD29-FITC, CD164-PE and CD44-APC, and one dim panel, CD49d-FITC, CD29-PE, and CD182-APC to ensure that the brightness of placeholder antibodies did not influence the core panel performance.

### Antibody Titration

Negative MSC marker antibodies (CD14, CD19, and CD45) were titrated on peripheral blood. CD34 was titrated on umbilical cord blood using the ISHAGE procedure to detect CD34+ cells [20]. Positive MSC marker antibodies (CD29, CD44, CD49d, CD73, CD90, CD105, CD164 and CD182) were titrated on UCM MSCs.

### Monoclonal Antibodies Staining Strategy

The panel was built in stages to ensure that the reagents could be used in combination. Initially UCM MSCs were single-stained with antibodies to the positive markers CD73, CD90 and CD105, double-stained in all three possible combinations, and then triple-stained. The results were analyzed to compare the signal for all combinations. Samples were then stained with the bright 10-colour panel (UCM MSC, n=3; BM MSC, n=3) or the dim 10-colour panel (UCM MSC, n=6; BM MSC, n=4). Antibody capture (AbC) beads (Life Technologies) were used to create compensation controls for all reagents. Fluorescence-minus-one (FMO) controls [21] were created in parallel by staining with all antibodies except for one in all 10 combinations to identify where to set the gates. UCM MSC samples (n=4) were mixed with HSF 1184 or PBMCs to ensure that MSCs could be identified as rare events in a mixed cell population.

### Monoclonal Antibodies Staining Conditions

Cells (3×105) in 100 μl FACS buffer (Dulbecco's PBS, Life Technologies; 0.2% BSA and 0.05% sodium azide, both Sigma Aldrich) were incubated on ice in the dark for 30 min with the appropriate amount of each antibody as determined by previous titration experiments. The cells were then pelleted by centrifugation at 515×g for 7 min at 4° C., the supernatant was discarded and the cells re-suspended in 3 ml of the same buffer, and pelleted again by centrifugation. After swiftly pouring off the supernatant the cell pellet was re-suspended by gently tapping the tube in the small amount of buffer which was left in the tube. The mixed population samples using fibroblasts and PBMCs were prepared so that the final cell number was 3×105 before staining.

### Flow Cytometry Data Acquisition

The stained cells were analyzed within 2 h using a BD FACSAria I flow cytometer (BD Bioscience) with FACS Diva 6.1.3 software. The filter configuration used is described in Table 2. The instrument was turned on for at least 1 h prior to each run to allow the lasers to warm up and Cytometer Setup & Tracking Beads (BD Bioscience) were used to check instrument performance. All samples were recorded without compensation. A minimum of 10,000 cell events were recorded for each monoclonal antibody combination. During the data acquisition of the mixed cell populations, 60,000 cell events were recorded for the fibroblasts and 100,000 events for the PBMCs. Voltages were set on unstained samples [22].

### Flow Cytometry Data Analysis

The FCS files were automatically compensated and analyzed in Kaluza 1.2 (Beckman Coulter) using the data from the AbC beads to create the compensation rules. Throughout the study median fluorescent intensity (MFI) was displayed on logicle (bi-exponential) axes to enable visualization of negative events below the axes [23]. To convey information as to the density of events, contour density plots with visualized outliers were chosen as the standard plot [24]. The median fluorescence intensity (MFI) from the whole cell population was obtained. Descriptive statistics included medians, lowest and highest values. Statistical significance was assessed by student's t-test.

### Results

### Gating Strategy to Identify MSCs

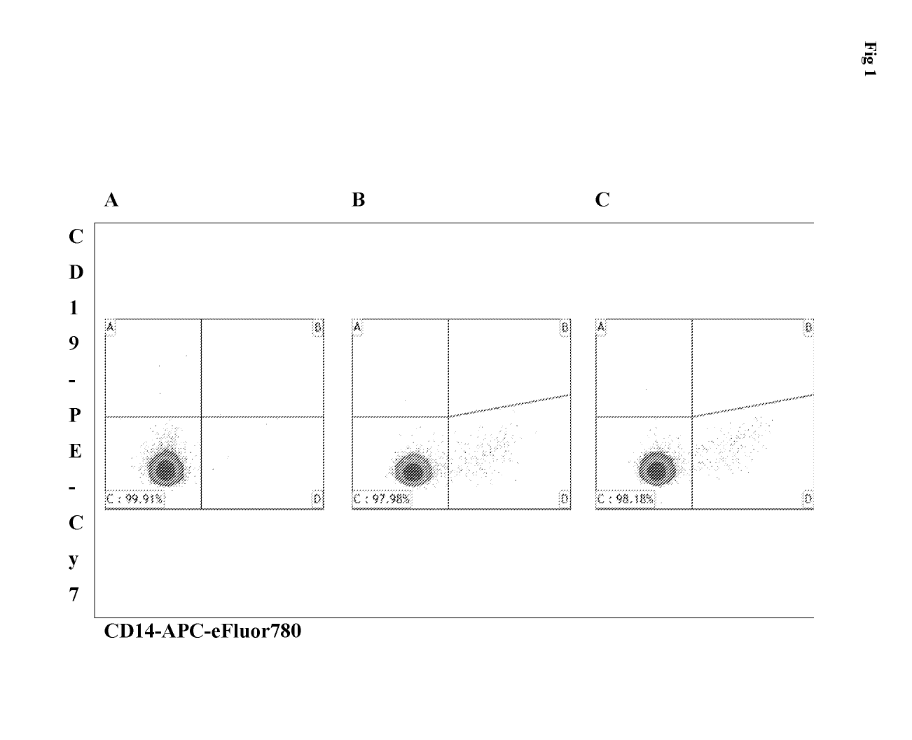

All the gates were set using fluorescence minus one (FMO) controls to ensure accurate gating (FIG. 1) [21]. To exemplify, the CD14-FMO was displayed on a CD14 vs. CD19 plot with a quadrant gate. The CD14 gate was adjusted until <1% of the events fell into the right quadrants (A). Then the CD19-FMO sample was displayed on the same plot and the gates were adjusted for CD19 accordingly (B). Lastly, the sample stained with all antibodies was visualized using the set gates (C). The same procedure was carried out for all FMOs.

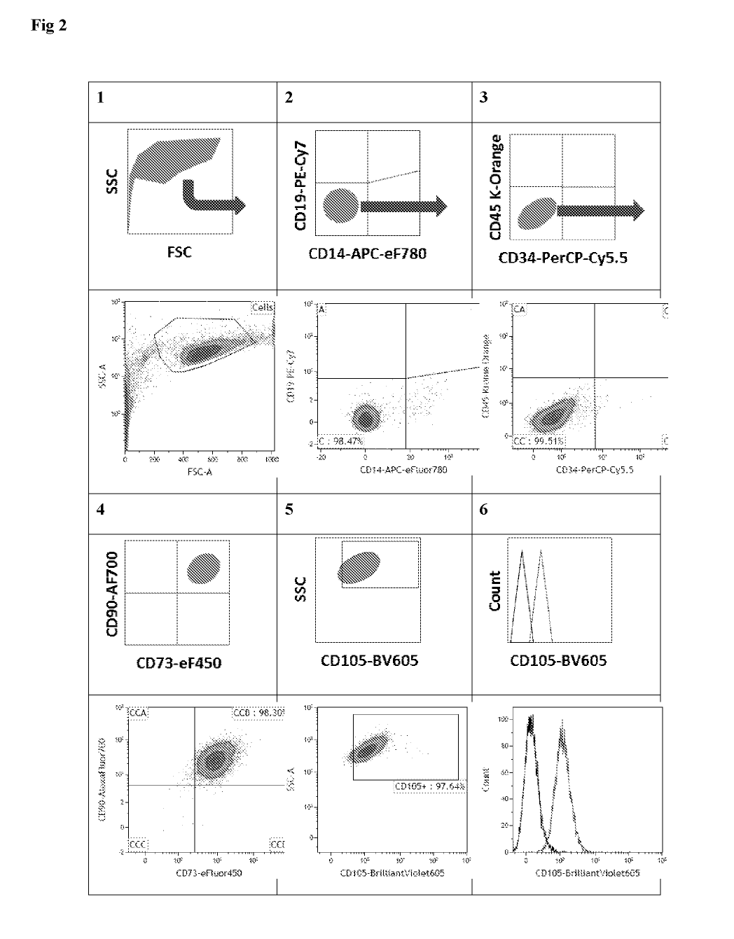

Using the gates set with the FMOs a multi-step gating strategy was developed to identify MSCs, schematically outlined and exemplified with UCM MSCs in FIG. 2. First, all recorded events were visualized on a forward scatter vs. side scatter plot and a gate was drawn around the cells to exclude debris and doublets (Step 1). Side scatter was displayed on a logarithmic axis and forward scatter on a linear axis. The gated cells were displayed on a CD14 vs. CD19 plot to identify double-negative (CD14−/CD19−) events (Step 2). CD14−/CD19− events were displayed on a CD34 vs. CD45 plot and the CD34−/CD45− events were gated (Step 3). These were displayed on a CD73 vs. CD90 plot and the double-positive (CD73+/CD90+) events were gated (Step 4). The double-positive events were displayed on a CD105 vs. side scatter plot to view the CD105 intensity (Step 5). Of the positive markers, CD105 intensity was found to vary most between samples. As CD73 and CD90 were consistently bright these were used prior to CD105 in the gating strategy. Overlaying the histogram for CD73+/CD90+/CD105+ cells over that for unstained cells confirmed that the whole population was CD105+(Step 6). There was no difference between the CD105 FMO and the unstained cells in CD105 MFI wherefore the unstained control could be used.

### Phenotype Analysis

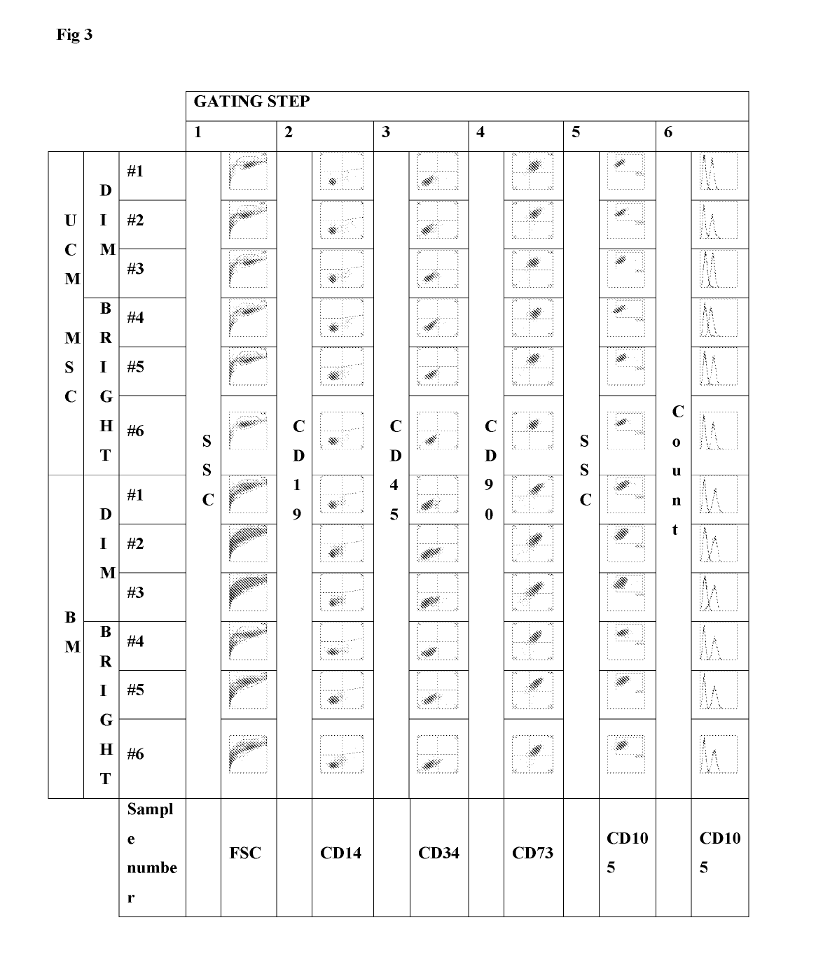

The core surface marker pattern displayed through the gating strategy was consistent for BM and UCM MSCs, and was not affected by the signal intensity of the placeholders (FIG. 3). The UCM MSCs displayed a more homogenous forward and side scatter profile. The heterogeneity seen outside the BM MSC cell gate in samples #2 and #3 is most likely cellular debris, reflecting their lower viability (82% and 86%, respectively). BM MSCs isolated from BM aspirates from trauma patients display a small CD34dim population (samples #2, #3, #5 and #6) compared to commercial BM aspirate (sample #4), commercial BM MSCs (#1), and UCM MSCs.

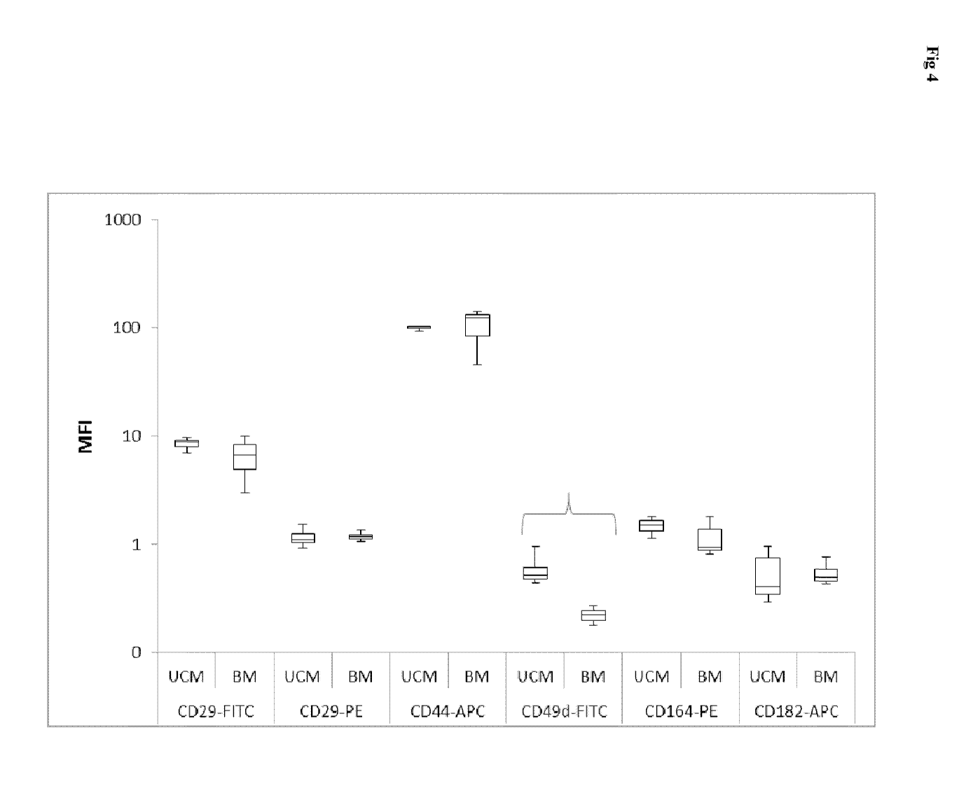

Intensity Analysis of Surface Antigens on Bone Marrow vs. Umbilical Cord Matrix MSCs There was no statistically significant difference between the BM and UCM MSCs in terms of MFI for positive and negative ISCT markers (data not shown). With the bright panel CD14 and CD34 had MFIs higher than the unstained control which indicates the importance of using FMOs to set the gates to exclude false positives. CD49d MFI was higher in UCM MSCs than BM MSCs (p=0.006) (FIG. 4). CD44 showed a broader range in signal strength in BM MSCs compared to UCM MSCs. The dim and bright panel did reveal that the expression of an antigen, in this case CD29, is very dependent on clone and/or fluorochrome used: CD29-FITC gave a higher MFI than CD29-PE when used to stain the same samples.

### Identifying MSCs in a Mixed Cell Population

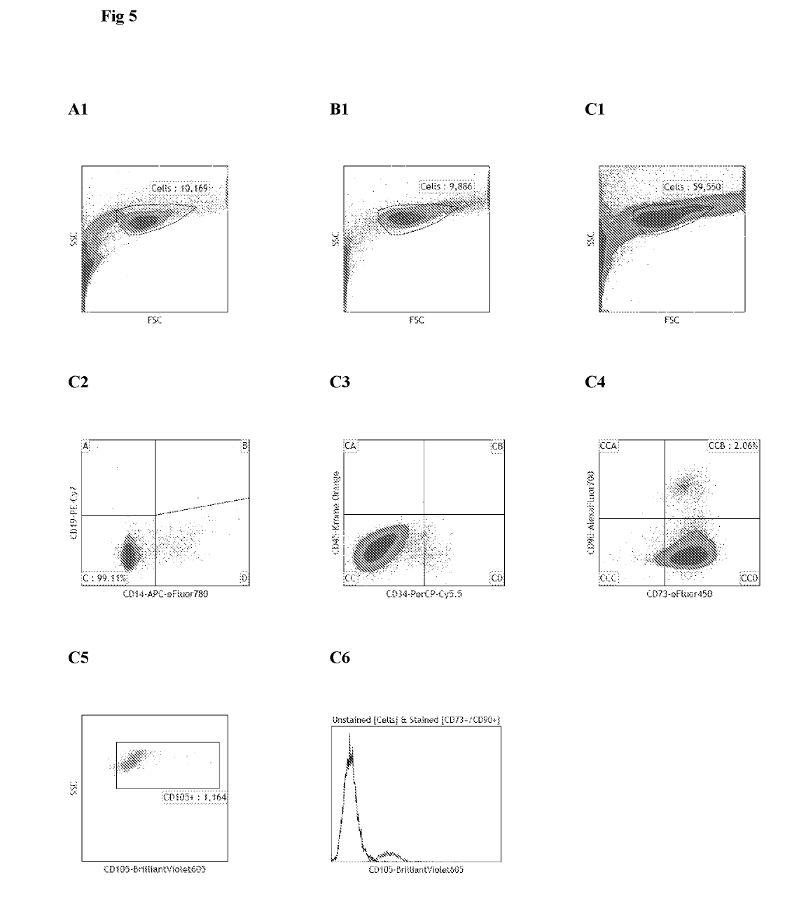

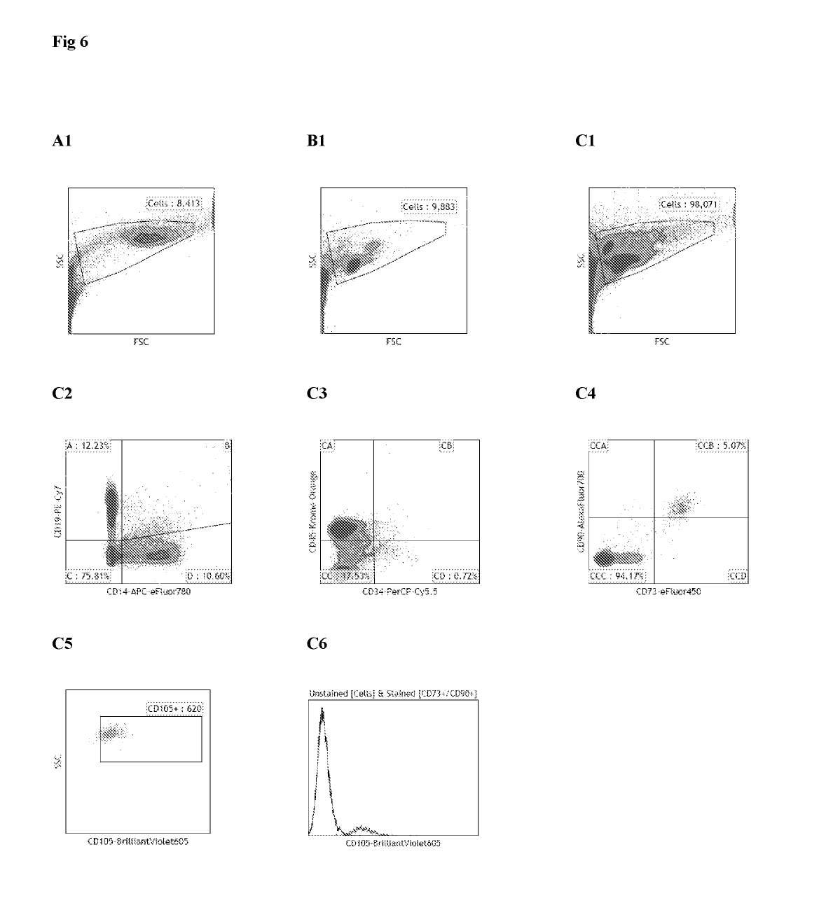

MSCs could be identified when mixed with a population of fibroblasts at 2% (2.0%, 1,164 MSCs out of 59,550 cell events), 5% (4.6%, 2730 MSCs out of 59,587 cell events) and 10% (10.4%, 6193 MSCs out of 59,474 cell events) (FIG. 5), repeated twice. It was also possible to identify MSCs added to PBMCs at 1% (0.63%, 620 MSCs out of 98,071 cell events), 2% (1.9%, 1860 MSCs out of 97,705 cell events), 5% (3.7%, 3627 MSCs out of 97,360 cell events) and 10% (6.8%, 6535 MSCs out of 95,772 cell events) (FIG. 6), performed once. However, PBMC preparations are dominated by cells that express the negative ISCT MSC markers—CD14, CD19, CD34, and CD45. Therefore, the gating strategy had to be modified with FMOs for these markers re-calculated on MNCs positive for their expression. The cultured MSCs also have higher autofluorescence than the primary PBMCs in the violet channel, causing some MSCs to appear false positive for CD45, whereby they are excluded in gating step 3. This caused a slight reduction in the identification rate.

### CONCLUSIONS

MSCs are a rare cell population in vivo, and MFC enables their identification and study. To exemplify this, MSCs were mixed with HSFs and with PBMCs at low concentrations (1-10%), and it was possible to detect them using this panel. The surface marker expression of fibroblasts is similar to MSCs [25] and they are present in many of the same tissues. Fibroblasts are a common cell type in several tissues. They have been shown to share the ISCT-markers [25] and to differentiate into adipocytes, chondrocytes and osteocytes [26]. Therefore, in the cases when it is important to isolate a pure starting material of MSCs from primary tissues, such as for clinical use, it is desirable to be able to distinguish these two cell types. In this study, the human skin fibroblast cell line used was CD90-negative which enabled cell separation based on this marker. However, as primary fibroblasts have been shown to be CD90+, placeholders that better distinguish fibroblasts using described positive markers of these cells, such as CD10, CD29, and CD106 [25], could be used.

PBMCs consist mostly of lymphocytes and monocytes, mixing MSCs with these is taking a step closer to identifying MSCs in bone marrow, blood, and other complex tissue samples. As the panel can be used to detect MSCs in this heterogeneous sample it could be used to re-evaluate studies of circulating MSCs in various disease states that have been performed with 3-4 colour analysis and/or have not included ISCT-markers [27-31]. The panel could be used to quantify MSCs/μl sample either by a direct single platform method utilising quantification beads or indirectly using a double platform method combining the panel data with an automated haematology leukocyte count as used for CD34+ cell quantification [20]. This panel could thus be used to evaluate non-bone marrow MSC sources in a standardized manner.

As could be seen with the two CD29-conjugates in this study (FIG. 4), different clones and conjugates can produce very different intensity signals. It is important to identify an antibody of optimal performance for the intended study as performance can vary [32]. The bright and dim panels did not affect the performance of the 7-colour core panel. When modifying the panel for other purposes, the cells should be stained with the 7-colour core panel, each additional placeholder antibody (or functionality stain) separately, and then in combination to ensure that the addition does not influence the core panel results.

Inaccuracies in the ability to measure the signal from the cells, related to the error arising from photon-counting statistics, this error can result in a spread of the population where even data statistically compensated by conventional means can produce false data events that may appear positive [33]. This “spreading error” is most noticeable with far-red dyes such as the APC-eFluor780 used to detect CD14 in this panel. Thus, when using this panel it is important to display data using logicle axes to adjust for the spread, and to use a CD14-FMO to set the threshold. Spreading error can also be compensated by using an aggregated classification and regression tree model formed using a bootstrap algorithm including a combination of many distinct trees, each model estimated in a sequence of bootstrap samples drawn from the original sample, herein a screening decision is used to calculated the phenotype of MSC cells based on a combination of average predicted favorable across bootstrap trees and a majority vote criterion comprising whether a majority of the bootstrap trees predict favorable above a predetermined threshold level. To conclusion, the innovative 10-colour panel and algorithm proposed in this application consisting of a statistical modeling program and 7 ISCT core-panel markers and can even include up to 3 placeholder positions to be populated as required, they can be used to phenotype bone marrow and umbilical cord MSCs. It provides a flexible flow cytometry tool for researchers in any aspect of MSC research to study MSCs and their subtypes in combination with functional cell dyes for visualization of events such as apoptosis, cell cycle analysis and proliferation on a cell-by-cell basis.

### REFERENCES

1. Chen, S.-I., et al., Effect on left ventricular function of intracoronary transplantation of autologous bone marrow mesenchymal stem cell in patients with acute myocardial infarction. Am J Cardiol, 2004. 94: p. 92-95.
2. Hare, J. M., et al., A randomized, double-blind, placebo-controlled, dose-escalation study of intravenous adult human mesenchymal stem cells (prochymal) after acute myocardial infarction. J Am Coll Cardiol, 2009. 54(24): p. 2277-86.
3. Bang, O. Y., et al., Autologous mesenchymal stem cell transplantation in stroke patients. Ann Neurol, 2005. 57(6): p. 874-82.
4. Lee, P. H., et al., Autologous mesenchymal stem cell therapy delays the progression of neurological deficits in patients with multiple system atrophy. Clin Pharmacol Ther, 2008. 83(5): p. 723-30.
5. Le Blanc, K., et al., Mesenchymal stem cells for treatment of steroid-resistant, severe, acute graft-versus-host disease: a phase II study. Lancet, 2008. 371(9624): p. 1579-86.
6. Kang, K. S., et al., A 37-year-old spinal cord-injured female patient, transplanted of multipotent stem cells from human UC blood, with improved sensory perception and mobility, both functionally and morphologically: a case study. Cytotherapy, 2005. 7(4): p. 368-73.
7. Dominici, M., et al., Minimal criteria for defining multipotent mesenchymal stromal cells. The International Society for Cellular Therapy position statement. Cytotherapy, 2006. 8(4): p. 315-317.
8. De Rosa, S. C., J. M. Brenchley, and M. Roederer, Beyond six colors: a new era in flow cytometry. Nat Med, 2003. 9(1): p. 112-7.
9. Martins, A. A., et al., Quantification and immunophenotypic characterization of bone marrow and umbilical cord blood mesenchymal stem cells by multicolor flow cytometry. Transplantation Proceedings, 2009. 41: p. 943-946.
10. Jones, E. A., et al., Optimization of a flow cytometry-based protocol for detection and phenotypic characterization of multipotent mesenchymal stromal cells from human bone marrow. Cytometry part B (clinical cytometry), 2006. 70B: p. 391-399.
11. Tucker, H. A. and B. A. Bunnell, Characterization of human adipose-derived stem cells using flow cytometry. Methods Mol Biol, 2011. 702: p. 121-31.
12. Bradford, J. A. and S. T. Clarke, Panel development for multicolor flow-cytometry testing of proliferation and immunophenotype in hMSCs. Methods Mol Biol, 2011. 698: p. 367-85.
13. La Rocca, G., et al., Isolation and characterization of Oct-4+/HLA-G+ mesenchymal stem cells from human umbilical cord matrix: differentiation potential and detection of new markers. Histochem Cell Biol, 2009. 131(2): p. 267-82.
14. Najar, M., et al., Mesenchymal stromal cells use PGE2 to modulate activation and proliferation of lymphocyte subsets: Combined comparison of adipose tissue, Wharton's Jelly and bone marrow sources. Cell Immunol, 2010. 264(2): p. 171-9.
15. Ohgushi, H., et al., Tissue engineered ceramic artificial joint-ex vivo osteogenic differentiation of patient mesenchymal cells on total ankle joints for treatment of osteoarthritis. Biomaterials, 2005. 26(22): p. 4654-4661.
16. Wexler, S. A., et al., Adult bone marrow is a rich source of human mesenchymal ‘stem’ cells but umbilical cord and mobilized adult blood are not. Br J Haematol, 2003. 121(2): p. 368-74.
17. Gronthos, S., et al., Surface protein characterization of human adipose tissue-derived stromal cells. Journal of cellular physiology, 2001. 189(1): p. 54-63.
18. Biihring, H. J., et al., Phenotypic characterization of distinct human bone marrow-derived MSC subsets. Annals of the New York Academy of Sciences, 2009. 1176(1): p. 124-134.
19. Ringe, J., et al., Towards in situ tissue repair: human mesenchymal stem cells express chemokine receptors CXCR1, CXCR2 and CCR2, and migrate upon stimulation with CXCL8 but not CCL2. J Cell Biochem, 2007. 101(1): p. 135-46.
20. Barnett, D., et al., Guideline for the flow cytometric enumeration of CD34+ haematopoietic stem cells PREPARED BY THE CD34+HAEMATOPOIETIC STEM CELL WORKING PARTY*. Clinical & Laboratory Haematology, 1999. 21(5): p. 301-308.
21. Roederer, M., Compensation in flow cytometry. Curr Protoc Cytom, 2002. Chapter 1: p. Unit 114.
22. Maecker, H. T. and J. Trotter, Flow cytometry controls, instrument setup, and the determination of positivity. Cytometry A, 2006. 69(9): p. 1037-42.
23. Herzenberg, L. A., et al., Interpreting flow cytometry data: a guide for the perplexed. Nat Immunol, 2006. 7(7): p. 681-5.
24. Alvarez, D. F., et al., Publishing flow cytometry data. Am J Physiol Lung Cell Mol Physiol, 2010. 298(2): p. L127-30.
25. Cappellesso-Fleury, S., et al., Human fibroblasts share immunosuppressive properties with bone marrow mesenchymal stem cells. Journal of clinical immunology, 2010. 30(4): p. 607-619.
26. Lysy, P. A., et al., Human skin fibroblasts: From mesodermal to hepatocyte-like differentiation. Hepatology, 2007. 46(5): p. 1574-85.
27. Wang, Y., et al., Changes in circulating mesenchymal stem cells, stem cell homing factor, and vascular growth factors in patients with acute ST elevation myocardial infarction treated with primary percutaneous coronary intervention. Heart, 2006. 92(6): p. 768-74.
28. Mansilla, E., et al., Bloodstream cells phenotypically identical to human mesenchymal bone marrow stem cells circulate in large amounts under the influence of acute large skin damage: new evidence for their use in regenerative medicine. Transplantation Proceedings, 2006. 36: p. 967-969.
29. Villaron, E. M., et al., Mesenchymal stem cells are present in peripheral blood and can engraft after allogeneic hematopoietic stem cell transplantation. haematologica, 2004. 89(12): p. 1421-7.
30. Fernandez, M., et al., Detection of stromal cells in peripheral blood progenitor cell collections from breast cancer patients. Bone Marrow Transplant, 1997. 20(4): p. 265-71.
31. Bian, Z. Y., et al., Increased number of mesenchymal stem cell-like cells in peripheral blood of patients with bone sarcomas. Arch Med Res, 2009. 40(3): p. 163-8.
32. Mahnke, Y. D. and M. Roederer, Optimizing a multicolor immunophenotyping assay. Clin Lab Med, 2007. 27(3): p. 469-85, v.
33. Roederer, M., Multiparameter FACS analysis. Curr Protoc Immunol, 2002. Chapter 5: p. Unit 58.

### Tables

TABLE 1

| | Conjugates for | | | |
|---|---|---|---|---|
| | CD11 | | | CD79 |
| Supplier | b | CD14 | CD19 | α |
| Ab Cam | 10 | 12 | 12 | 3 |
| Beckman Coulter | 5 | 8 | 10 | 2 |
| BD Bioscience | 12 | 13 | 16 | 2 |
| BioLegend | 16 | 15 | 15 | 3 |
| eBioscience | 9 | 11 | 13 | 4 |
| Life Technologies | 4 | 8 | 15 | 2 |
| Merck Millipore | 3 | 3 | 2 | 1 |
| Miltenyi Biotec | 7 | 9 | 7 | 0 |
| RnD Systems | 4 | 5 | 5 | 3 |
| Santa Cruz | 0 | 4 | 2 | 0 |
| Biotechnology | | | | |
| AbD Serotec | 7 | 9 | 8 | 4 |
| Sigma Aldrich | 2 | 3 | 3 | 2 |
| Total Conjugates | 79 | 100 | 108 | 26 |
| Available | | | | |

### Evaluation of Number of Antibody Conjugates Per Monocyte Marker (CD11b and CD14) and Per B-Cell Marker (CD19 and CD79a). Search Performed in July 2012. The 13^{th} Supplier, ImmuQuest, Had No Suitable Products Available.

TABLE 2
FACS Aria I Filter Configuration

| | | Long pass | Band pass | Emission | | |
|---|---|---|---|---|---|---|
| Laser | Detector | filter | filter | Range | Fluorochrome | Marker |
| Violet | A | 600 | 610/20 | 600-620 nm | BV605 | CD105 |
| (405 nm) | B | 502 | 530/30 | 515-545 nm | K-Orange | CD45 |
| | C | None | 450/40 | 425-475 nm | eF450 | CD73 |
| Blue | A | 735 | 780/60 | 750-810 nm | PE-Cy7 | CD19 |
| (488 nm) | B | 655 | 695/40 | 675-715 nm | PerCP-Cy5.5 | CD34 |
| | C | 595 | 610/20 | 600-620 nm | — | — |
| | D | 556 | 585/42 | 564-606 nm | PE | Placeholder |
| | E | 502 | 530/30 | 515-545 nm | FITC/AF488 | Placeholder |
| | F | — | 488/10 | 483-493 nm | — | — |
| | G | — | — | — | — | — |
| | H | — | — | — | — | — |
| Red | A | 735 | 780/60 | 750-810 nm | APC-eF780 | CD14 |
| (633 nm) | B | 691 | 710/40 | 690-730 nm | AF700 | CD90 |
| | C | — | 660/20 | 650-670 nm | APC | Placeholder |

1. (canceled)

2. A method of identifying a specific cell type in a population of cells, comprising simultaneously identifying using FACS the presence or absence of seven or more cell surface markers indicative of the specific cell type on the surfaces of the cells in the population.

3. A method according to claim 2, wherein the method comprises identifying the presence or absence of ten or more cell surface markers indicative of the specific cell type on the surfaces of the cells in the population.

4. A method according to claim 2, wherein the specific cell type is a mesenchymal stem cell (MSC), wherein the method comprises simultaneously identifying the presence or absence of CD73, CD90, CD105, CD14, CD19, CD34 and CD45 and wherein the presence of detectable levels of CD73, CD90 and CD105 and the absence of detectable levels of CD14, CD19, CD34 and CD45 on the surface of a cell indicates that it is a MSC.

5. A method according to claim 4, wherein the method further comprises identifying the presence or absence of detectable levels of CD181 and CD184 and wherein the presence of detectable levels of CD181 and C184 on the surface of a cell indicates that it is a particular subtype of MSC.

6. A method according to claim 2, wherein the specific cell type is a progenitor cell of mesodermal lineage, wherein the method comprises identifying the presence or absence of CD29, CD44, CD73, CD90, CD105, CD271, CD14, CD34 and CD45 and wherein the presence of detectable levels of CD29, CD44, CD73, CD90, CD105 and CD271 and the absence of detectable levels of CD14, CD34 and CD45 on the surface of a cell indicates that it is a progenitor cell of mesodermal lineage.

7. A method according to claim 6, wherein the method further comprises identifying the presence or absence of C—X—C chemokine receptor type 1 (CXCR1), CXCR2 or CXCR4 and wherein the presence of detectable levels of CXCR1, CXCR2 or CXCR4 on the surface of a cell indicates that it is capable of migrating to a specific, damaged tissue in a patient.

8. A method according to claim 7, wherein the presence of detectable levels of CXCR4 on the surface of a cell indicates that it is capable of migrating to damaged cardiac tissue, retinal tissue or bone tissue in a patient.

9. A method according to claim 2, wherein:
(a) the specific cell type is a fibroblast, wherein the method comprises identifying the presence or absence of, amongst others, CD10, CD29 and CD106 and wherein the presence of detectable levels of CD10, CD29 and CD106 on the surface of a cell indicates that it is a fibroblast; or
(b) the specific cell type is a very small embryonic/epiblast-like stem cell (VSEL), wherein the method comprises identifying the presence or absence of detectable levels of Sca-1, CD45R, Gr-1, TCRalphabeta, TCRgammadelta, CD11b, Ter119 and Oct-4 and wherein the presence of detectable levels of Sca-1, CD45R, Gr-1, TCRalphabeta, TCRgammadelta, CD11b, Ter119 and Oct-4 on the surface of a cell indicates that it is a VESL; or
(c) the specific cell type is a hematopoietic stem cells (HSC), wherein the method comprises identifying the presence or absence of detectable levels of, amongst others, CD133 and wherein the presence of detectable levels of CD133 on the surface of a cell indicates that it is a HSC; or
(d) the specific cell type is a endothelial progenitor cell (EPC), wherein the method comprises identifying the presence or absence of detectable levels of, amongst others, KDR, VE-cadherin and CD31 and wherein the presence of detectable levels of KDR, VE-cadherin and CD31 on the surface of a cell indicates that it is a EPC; or
(e) the specific cell type is a tissue-committed stem cell, wherein the method comprises identifying the presence or absence of detectable levels of, amongst others, CD117+, CD184, c-met and AC133 and wherein the presence of detectable levels of CD117+, CD184, c-met and AC133 on the surface of a cell indicates that it is a tissue-committed stem cell.

10-14. (canceled)

15. A kit for identifying a specific cell type in a population of cells, comprising seven or more fluorescently-labelled antibodies, wherein at least one antibody in the kit specifically binds to one of seven or more cell surface markers indicative of the specific cell type.

16. A kit according to claim 15, wherein the kit comprises ten or more fluorescently-labelled antibodies, wherein at least one antibody in the kit specifically binds to one of ten or more cell surface markers indicative of the specific cell type.

17. A kit according to claim 15, wherein at least one antibody in the kit specifically binds to:
(a) each of CD73, CD90, CD105, CD14, CD19, CD34 and CD45; or
(b) each of CD181 and CD184; or
(c) each of CD29, CD44, CD73, CD90, CD105, CD271, CD14, CD34 and CD45, and optionally to each of C—X—C chemokine receptor type 1 (CXCR1), CXCR2 and CXCR4; or
(d) each of CD10, CD29 and CD106; or
(e) each of Sca-1, CD45R, Gr-1, TCRalphabeta, TCRgammadelta, CD11b, Ter119 and Oct-4; or
(f) CD133; or
(g) each of KDR, VE-cadherin and CD31; or
(h) each of CD117+, CD184, c-met and AC133.

18-25. (canceled)

26. A reagent or composition of matter for:
(a) identifying by virtue of the relative specific concentrations of the active components of FAC reagent a mesenchymal stem cell (MSC) in a cell population, comprising simultaneously identifying using fluorescence activated cell sorting (FACS) a cell in the population which displays detectable levels of CD73, CD90 and CD105 and does not display detectable levels of CD14, CD19, CD34 and CD45 on its surface and thereby identifying a mesenchymal stem cell (MSC) in a cell population; or
(b) identifying by virtue of the relative specific concentrations of the active components of FAC reagent a specific cell type in a population of cells, comprising simultaneously identifying using FACS the presence or absence of seven or more cell surface markers indicative of the specific cell type on the surfaces of the cells in the population.

27. (canceled)

28. A method of producing a panel of fluorescently-labelled antibodies for simultaneously identifying the presence or absence of seven or more cell surface markers using FACS analysis, the method comprising (a) selecting the seven or more cell surface markers; (b) spreading the positive markers on different lasers; (c) selecting seven or more fluorescently-labelled antibodies; (d) titrating the antibodies to ensure that optimal concentration and minimal spectral overlap is achieved on cells with known expression of the seven or more markers (both positive and negative); (e) testing the antibodies against the positive cell surface markers; (f) testing a core panel of the antibodies; (g) inducing or stimulating cells to express the placeholder markers; (h) testing and titrating the placeholder antibodies; (i) optimising the placeholder conditions; and (j) testing the full panel on cells in the absence of other cell types and in mixed populations.

29. A panel of fluorescently-labelled antibodies for simultaneously identifying the presence or absence of seven or more cell surface markers using FACS analysis produced using the method of claim 28.

30. A method of identifying a novel cell type in a population of cells, comprising simultaneously identifying using FACS the presence or absence of seven or more cell surface markers on the surfaces of the cells in the population, wherein at least some of the seven or more markers are indicative of a specific cell type and at least one of the seven or more markers is not detectably expressed by the specific cell type.

31. A kit according to claim 15, wherein the specific cell type is a mesenchymal stem cell (MSC) and at least one antibody in the kit specifically binds to each of CD73, CD90, CD105, CD14, CD19, CD34 and CD45.
